(19) United States
(12) Patent Application Publication
(10) Pub. No.: US 2010/0268142 A1
(43) Pub. Date: Oct. 21, 2010

(54) ELBOW BRACE FOR PREVENTING OR ATTENUATING TENNIS ELBOW

(76) Inventor: Franklin H. Nyi, Torrance, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 12/425,329

(22) Filed: Apr. 16, 2009

(51) Int. Cl. A61F 5/00

(52) U.S. Cl. 602/20

(57) ABSTRACT

An elbow brace utilizes a pair of shock-absorbing elements that are placed over the arm over the lateral and medial epicondyle of the humerus bone. These elements provide a medium for absorbing a portion of the shock energy and vibration that can travel along the forearm and exit the elbow of a person during physical activity. Pressure transmitting elements are designate to localize pressure directly on the tendon that extends from the extensor digitorum muscle and is attached to the humerus. The other pressure transmitting element is placed over the tendon of the triceps brachii which is attached to the olecranon of the ulna. A positioning strap provides quick and easy ability to align the components on the arm of the wearer. Additional pressure generating straps help to generate focused pressure to the tendons and helps to maintain the brace on the arm of the wearer.

### BACKGROUND OF THE INVENTION

The present invention relates generally to arm and elbow braces and, more particularly, to an elbow brace for preventing or attenuating the painful inflammation of the tendons in the elbow, commonly known as “tennis elbow.”

A number of arm braces have been developed to treat and/or prevent a condition known as “tennis elbow.” Tennis elbow is a commonly used term to describe a painful, medical condition which is characterized by inflammation of the ligaments that connect the two bones of the forearm, the radius and ulna, and inflammation of the tendons of the muscles of the forearm that are attached to the two spurs of the humerus, the medial and lateral epicondyle. Inflammation can also occur to the tendon of the triceps brachii which is attached to the olecranon of the ulna and can also extend to the tissues in the area directly surrounding the medial and lateral epicondyle.

The most characteristic symptom of tennis elbow is extreme pain which occurs from continued participation in the physical activity. Pain can also be brought on through simple movement of the forearm, for example, when a person lifts even a light object. The pain resulting with tennis elbow has been most often attributed to the inflammation that occurs to the tendons of the muscles of the forearm which are attached to the medial and lateral epicondyle of the humerus bone. Also, due to the shock that can be generated in the elbow region during physical activities, inflammation to the surrounding tissues can occur and also cause pain. Inflammation of the tendon extending from the triceps brachii to the olecranon also adds to the overall pain suffered by the afflicted person.

The shock which travels along the forearm of the player when striking an object, such as a ball, with another object, such as a tennis racket, can cause at least some of the tendons attached at the epicondyle to stretch and move outward away from the bone in a phenomena sometimes referred to as “pouching.” This phenomenon is especially prevalent to the tendon of the extensor digitorum muscle which is attached to the epicondyle. Since this tendon is somewhat larger than most tendons in the area, the resulting shock and movement of the muscles during the swing of the player's arm causes the tendon to stretch and move outward away from the underlying bone. As a result, continued shock can cause this particular tendon to continue to stretch and “pouch” causing the tendon to enlarge and become inflamed. In some situations, the tendons become so stretched from its original size that surgery must be performed to reduce its size back to the size of a normal tendon. This, of course, is a rare occurrence but shows the damage that the shock energy can inflict on at least one of the tendons of the forearm. Similar pouching can also occur to the tendon extending from the triceps brachii which is in turn attached to the olecranon of the ulna. This particular tendon can also stretch and pouch resulting in inflammation and its associated discomfort. Prolonged and continuous subjection of this tendon to the shock wave can cause additional stretching and increased pain from inflammation.

My previous invention disclosed in U.S. Pat. No. 5,063,913, which is incorporated herein in its entirety, helps to prevent or alleviate tennis elbow through the application of direct pressure on various tendons that are susceptible to abnormal stretching during strenuous athletic activities, along with the placement of shock-absorbing elements around the epicondyle of the humerus bone to absorb and dissipate at least a portion of any shock energy that may travel along the forearm of the user. My previous invention reduces or prevents pouching from occurring which will help to prevent one from becoming inflicted with tennis elbow in the first place and will also help to dissipate pain and inflammation for those who already suffer from tennis elbow. The elbow brace which I previously developed utilizes a pair of shock-absorbing elements that are placed on the arm over the medial and lateral epicondyle of the humerus bone. These shock-absorbing elements can be made from an absorbent rubber, a rubber-like or similar shock-absorbing material, which can provide a means for absorbing the shock energy and vibration associated with the physical activity that can promote tennis elbow. This elbow brace also utilizes pressure transmitting elements designed to impart a localized and direct amount of pressure onto specific tendons that are more vulnerable to stretching and developing tennis elbow and its associated inflammation and discomfort. In one form of my previous invention, a pressure transmitting element is designed to localize pressure directly onto the tendon that extends from the extensor digitorum muscle and is attached to the humerus. In another form of the invention, another pressure transmitting element is placed in contact with the arm directly over the tendon of the triceps brachii which is attached to the olecranon of the ulna. These pressure transmitting elements provide a sufficient amount of radial force or pressure on these particular tendons to prevent them from pouching during physical activity. As a result, the trauma and stretching that occurs during the physical activity are diminished thus resulting in prevention or an attenuation of the condition known as tennis elbow.

Other known treatments to prevent tennis elbow utilize braces which simply apply pressure around the forearm of the player to prevent some of the movement of the muscles and tendons during the physical activity. Such devices usually are made from an elastic band and Velcro straps which apply a radial pressure to the forearm. In many devices, a generalized pressure is applied to the region of the elbow without much attention being given to localizing or focusing the pressure to the particular tendons that are more susceptible to the phenomenon of pouching. As a result, these prior art devices provide some means for preventing pouching, but for the most part, only reduce it a small amount. Also, prior art devices have been utilized to apply direct pressure onto the medial and lateral epicondyle to reduce the amount of stretching of the tendons in this region. These devices are somewhat helpful but most often do not provide nearly enough pressure, due to their design, to prevent trauma in this region. Prior art devices that place pressure on the epicondyle simply do not appreciate the effect of reducing or dissipating the shock energy which exits via the elbow.

The better approach is to localize or focus the pressure and increase it to prevent the tendons of interest from stretching or pouching, as is used in my previous invention. Also, it is preferable to somehow absorb the shock energy which exits via the elbow in order to reduce the amount of trauma that can be caused to the tendons and tissues in the areas surrounding the epicondyle. By merely pressing a hardened object against the epicondyle, or wrapping it with an elastic band, little, if any, shock is actually absorbed by these elements, thus ultimately diminishing their ability to alleviate trauma caused by the shock.

A person using an elbow brace made in accordance with my invention disclosed in U.S. Pat. No. 5,063,913 needs to carefully place the shock-absorbing elements and pressure transmitting elements on the proper locations of the tendons of concern. If these elements are not placed in proper contact with the tendons, then the effectiveness of my invention will be diminished. Therefore, there is a need for a device which will assist the user in properly placing the shock-absorbing elements and pressure transmitting elements in proper position on the wearer's arm. The composite elbow brace should also be designed so that the device can be simply placed on the arm and easily fixed into place allowing the various elements to perform their designated function with a minimal need to continually maneuver and maintain the elements at their proper location on the arm. The present invention satisfies these and other needs.

### SUMMARY OF THE INVENTION

The present invention provides a novel means for properly positioning and maintaining an elbow brace on the arm of its wearer. The present invention insures that the components designed to dissipates the shock energy that can be generated and travel along the forearm of the player will be placed at the properly location along the wearer's arm. Likewise, the present invention makes it easier for one to align the components used to place focused pressure to the tendons of interest at the right locations on the wearer's arm. As a result, these components, which play an important role in preventing or dissipating tennis elbow, will be properly placed on the wearer's arm to maximize the elbow brace's effectiveness.

In general terms, the present invention utilizes the same components described in my previous invention disclosed in U.S. Pat. No. 5,063,913, namely, a pair of shock-absorbing elements that are placed on the arm over the medial and lateral epicondyle of the humerus bone. These shock-absorbing elements are made from an absorbent rubber or rubber-like material, which can provide a means for absorbing the shock energy and vibration which are associated with the physical activity that can promote tennis elbow. The elbow brace further includes the same type of components used in my previous invention, namely, pressure transmitting elements designed to impart a localized and direct amount of pressure onto specific tendons that are more vulnerable to stretching and developing tennis elbow and its associated inflammation and discomfort. For example, one pressure transmitting element is designed to provide localize pressure directly onto the tendon that extends from the extensor digitorum muscle and is attached to the humerus. Another pressure transmitting element can be placed in contact with the arm directly over the tendon of the triceps brachii which is attached to the olecranon of the ulna. These pressure transmitting elements provide a sufficient amount of radial force or pressure on these particular tendons to prevent them from pouching during physical activity. As a result, the trauma and stretching that occurs during the physical activity are diminished thus resulting in prevention or an attenuation of the condition known as tennis elbow.

The present invention provides a novel and quick means for positioning these components at the proper locations on the arm of the wearer. The present invention includes a sleeve-like body forming a flexible, supporting material which houses and maintains the shock-absorbing elements and pressure transmitting elements on the properly location of the wearer's arm. In one embodiment, these shock-absorbing elements and pressure transmitting elements are placed on the inner surface of the sleeve-like body in pockets or pouches which are created on the body. The sleeve-like body includes a positioning strap utilized to allow the wearer to place the body in an “open” position wherein a portion of the sleeve-like body is opened to allow the wearer to visualize the exact location, for example, of the shock-absorbing components to permit the wearer to place the shock-absorbing components in direct contact with the medial and lateral epicondyle of the humerus bone. The positioning strap can be likewise be placed in a “closed” position in which the positioning strap is attached to the body to complete the encircling sleeve which extends around the wearer's arm. The positioning strap can be made with a quick release device, such as hook and loop fasteners, which allows the positioning strap to be quickly opened and closed by the wearer. This allows the wearer to quickly open and close the sleeve-like body multiple times, as needed, when positioning the shock-absorbing components and pressure transmitting elements on the arm. In this fashion, one can quickly open and close the sleeve-like body to properly position these components. If the components are still not properly aligned, the wearer can continue to open and close the sleeve-like body until he/she is satisfied with the placement of the shock-absorbing elements and pressure transmitting elements on the arm. In one embodiment, the positioning strap is placed at the end of the sleeve-like body where the pressure transmitting element contacts the upper arm of the wearer to prevent pouching of the tendon of the triceps brachii.

The present invention further includes strapping means associated with the sleeve-like body which are designed to generate and maintain an inward radial force or pressure on the wearer's arm. In one embodiment, a first pressure generating strap is disposed over the positioning strap. This first pressure generating strap applies an inward radial force to the upper arm of the wearer and likewise to the pressure transmitting element which helps to generate the focused pressure to the tendon of the triceps brachii. As a result, pouching of the tendon of the triceps brachii is greatly reduce or eliminated during the exercise. This first strap device is adjustable to produce the desired amount of radial force to the upper arm of the wearer. This allows the wearer to adjust the radial force as needed. A second pressure generating strap, similar to the above-described strap device, can be located at the other end of the sleeve-like body, to provide an inward radial force to the forearm of the wearer. This second pressure generating strap can be placed over the pressure transmitting element which prevents pouching of the tendon that extends from the extensor digitorum muscle and is attached to the humerus. Likewise, this second strap can be adjusted by the wearer to adjust the amount of radial force or pressure being generated on the forearm of the wearer. This second strap will also help generate the localized pressure which prevents the tendon from pouching.

In another particular form of the invention, the pressure transmitting elements may be comprised of generally elongate tubular members which extend approximately the length of the tendons that they contact. This particular structure is beneficial during play since the tubular members are generally disposed within pouches or pockets formed on the sleeve-like body which house the tubular members. The tubular members remain in place within the pockets during play and are maintained in proper contact with the region of the arm where the focused pressure is to be applied. Their configuration maintains them in direct contact with the tendons during play since the same amount of surface area contacts the tendon throughout play, even if the elements should rotate within the pocket. The tubular members can also be hollow to allow for some shock-absorbing capability during the physical activity.

In another embodiment of the present invention, a pair of pockets are disposed on the sleeve-like body to house the pressure transmitting element that prevents pouching of the tendon that extends from the extensor digitorum muscle and is attached to the humerus. A pair of side-by-side pockets are utilized for this particular pressure transmitting element since the location of this tendon varies from the left arm and right arm. For example, the wearer can place the pressure transmitting element in the pocket designated for the right arm when the elbow brace is to be worn on the right arm. The pressure transmitting element can be removed from the pocket designated for the right arm and placed into the adjacent pocket when the elbow brace is going to be worn on the wearer's left arm. In this regard, the elbow brace can be adapted for use on either the left or right arm of the wearer by simply placing the pressure transmitting element in the proper pocket.

The features and advantages of the present invention will become more apparent from the foregoing detailed description taken in conjunction with the accompanying drawings which illustrate by way of example the principle of the present invention.

### BRIEF DESCRIPTION OF THE DRAWINGS

FIG. 1 is a perspective view of one embodiment of an elbow brace made in accordance with the present invention as it is being placed on the arm of a user;

FIG. 2 is another perspective view of the elbow brace depicted in FIG. 1 as it is properly placed on the arm of a user;

FIG. 3 is a view of the elbow brace depicted in FIG. 1 showing the inner surface of the sleeve-like body which houses both the pressure transmitting elements and shock-absorbing elements;

FIGS. 4-6 shows one of the pressure transmitting elements being removed from one of the pockets and being placed in an adjacent pocket to allow the elbow device to be worn on either the right or left arm of the wearer;

FIG. 7 is a cross-sectional view showing a pressure transmitting element contained within a pocket formed on the sleeve-like body; and

FIG. 8 is a cross-sectional view of one of the shock-absorbing elements contained within a pocket formed on the sleeve-like body.

### DETAILED DESCRIPTION OF THE PREFERRED EMBODIMENTS

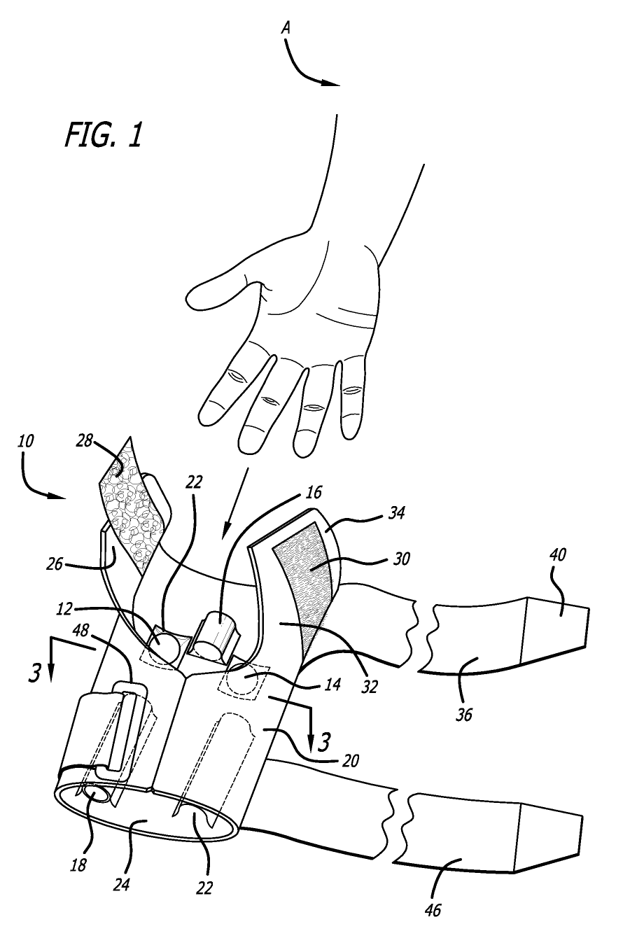

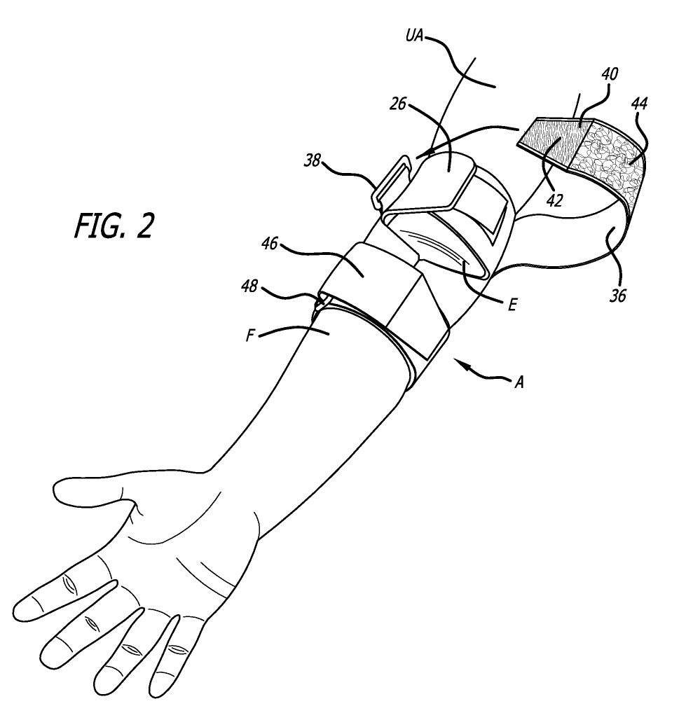

FIGS. 1 and 2 show one particular embodiment of an elbow brace made in accordance with the present invention. In FIG. 1, the elbow brace 10 is shown as the arm A of the wearer is initially being inserted into the device. FIG. 2 shows the same elbow brace 10 fully placed on the arm of the wearer. As can be seen in FIG. 2, the elbow brace 10 covers the forearm F, elbow region E, and upper arm UA of the wearer. FIG. 1 shows in phantom lines the various elements designed to impart the pressure onto the various tendons of the arm along with the elements which provide the shock-absorbing features that absorbs a good portion of the shock energy in the elbow region E in order to reduce the inflammation of the tendons at the epicondyle.

The elbow brace 10 shown in FIG. 1 includes a first shock-absorbing element 12 which comes in contact with the arm and is placed directly over the medial epicondyle of the humerus bone. A second shock-absorbing element 14 is also located on the elbow brace 10 and comes in contact with the arm and located directly over the lateral epicondyle of the humerus bone. FIGS. 1 and 2 do not show either the medial or epicondyle bone in the arm, however, it must be appreciated that these shock-absorbing components are placed directly over the specific regions in the arm to provide the shock-absorbing capabilities necessary to dissipate the trauma and inflammation caused by the shock energy which travels to and exits the elbow region E.

The elbow brace 10 includes a first pressure transmitting element 16 which is placed on the elbow brace 10 on the back side of the upper arm UA where it contacts the arm directly over the tendon of the triceps brachii which is attached to the ulna bone. A second pressure transmitting element 18 contacts the forearm F of the wearer and is placed directly over the tendon of the extensor digitorum muscle which is attached to the humerus. Again, FIGS. 1 and 2 do not show either of these tendons in the arm, however, it must be appreciated that these components are placed directly over these specific tendons in the arm to provide the focused pressure needed to prevent pouching of the tendons.

The elbow brace 10 can be manufactured as an elastic sleeve-like body 20 which also helps impart a slight inward radial pressure on the arm. This sleeve-like body 20 can also help impart some pressure onto the outer surface of the first and second shock-absorbing elements to apply a small amount of pressure to the tendons in the area of the medial and lateral epicondyle area. The elastic nature of the sleeve-like body 20 can also help maintain the various elements in direct contact with the arm to enable the elements to perform their particular functions. It should be noted that in FIGS. 1, 2 and 3, pouches or pockets 22 are shown formed on the inner surface 24 of the sleeve-like body to house these particular elements 12, 14, 16 and 18. It should be appreciated that the sleeve-like body 20 can be made, for example, from a double piece of fabric which has stitching which provides and creates the internal pockets 22. Other arrangements could be made in which individual pockets, as shown in FIG. 1, are formed on the inner surface 22 of the body 20. Clearly, there are any number of different possibilities that can be utilized in creating the particular means for holding and maintaining the shock-absorbing and pressure transmitting elements at the respective locations on the sleeve-like body 20.

In the particular embodiment disclosed in FIGS. 1 and 2, the sleeve-like body 20 includes a positioning strap 26 used by the wearer to properly position the shock-absorbing elements 12 and 14 on the elbow E. In this regard, the positioning strap 26 actually completes the tubular sleeve which encircles the wearer's upper arm UA and elbow region E. This positioning strap 26 includes a quick release fastener to allow the user to quickly open and close the strap 26 when attempting to position the elbow brace on the wearer's arm. For example, a hook-and-loop fastener can be used for quick release. As can be seen in FIG. 1, the positioning strap 26 has the loop portion 28 of the fastener attached thereto. The hook portion 30 of the fastener is, in turn, attached to an adjacent location 32 on the sleeve-like body. In the particular embodiment of FIGS. 1 and 2, this adjacent location of the sleeve-like body is formed as a strap 34 which cooperates with the positioning strap 26 to complete the encircling sleeve.

Initially, when the wearer first starts to pull the elbow brace 10 onto his/her arm, the location of the shock-absorbing elements 12 and 14 may not be exactly in contact with the medial and lateral epicondyle area. When the pressure transmitting elements 16 and 18 are not properly aligned over the tendons of interest, the positioning strap 26 allows the wearer to “open” a portion of the sleeve-like body 20 to allow the wearer to visualize where the shock-absorbing elements 12 and 14 are located on the inner surface 22 of the sleeve-like body. The opening of the body also allows the wearer to locate the medial or lateral epicondyle to allow the wearer to move the body 20, as needed, to place the shock-absorbing element over the area of the medial or lateral epicondyle. The wearer can then move the positioning strap back to a “closed” position in which the positioning strap 26 attaches to the second positioning strap 34. This will complete the sleeve which encircles the arm of the wearer and will help to prevent the elbow brace from moving on the arm of the wearer. If the shock-absorbing components 12 and 14 are still not in proper position over the distal and medial epicondyle region, the wearer can quickly open the positioning strap 26 and again attempt to reposition the shock-absorbing components. The use of the quick release fastener on the positioning strap 26 allows the wearer to quickly open and close the sleeve multiple times, if needed. It should be appreciated that the shock-absorbing elements 12 and 14 and the pressure transmitting elements 16 and 18 are disposed on the sleeve-like body such that proper alignment of only one of these elements will most likely cause the remaining elements to align properly on the arm as well. For this reason, proper alignment of the shock-absorbing elements 12 and 14 on the arm will likewise cause the pressure transmitting elements 16 and 18 to align properly on the arm as well.

The elbow brace 10 further includes means for applying an inward radial force to the arm of the wearer. As can be seen in FIGS. 1 and 2, a first pressure generating strap 36 is shown disposed over the positioning strap 26. This first pressure generating strap 36 applies a radial pressure which acts on the pressure transmitting element 16 which then creates the focused pressure that is applied to the tendon. This first pressure generating strap 36 also acts to help maintain the elbow brace at the desired location on the wearer's arm. This pressure generating strap 36 includes a quick release fastener, such as a hook-and-loop fastener, which allows the user to quickly and easily adjust the position of the strap 36 on the arm. This quick release fastener will also allow the wearer to adjust the amount of pressure being generated by the strap 36. As can be seen best in FIG. 2, the strap 36 includes a buckle 38 which is attached to outer surface of the sleeve-like body 20. The end 40 of the strap 36 can be placed through this buckle 38 and cinched back onto itself for attachment. As can be seen in FIG. 2, the end 40 of the strap 36 has the hook portion 42 of the quick release fastener attached to it. The end 40 of the strap 36 can then be attached to the loop portion 44 which extends along the length of the strap 36. The force or pressure generated by this strap 36 can be varied by changing the location where the end 40 is attached to the loop portion 44 of the strap 36.

A second pressure generating strap 46 also can be incorporated into the elbow brace 10 to better hold the device on the arm of the wearer. This second pressure generating strap 46 can also includes a buckle 48 and quick release fastener as described above with respect to the strap 36. This second strap 46 can be place at the end of the sleeve-like body 20 to apply pressure over the forearm F of the wearer. By positioning this second strap 46 over the pressure transmitting element 18, it will apply pressure to the second pressure transmitting element 18 which will in turn create the focused pressure that is to be applied to the tendon of the forearm. The amount of pressure generated by this second strap 46 can be adjusted accordingly. This second strap 46 will also help to maintain the elbow brace 10 at the desired location of the arm of the wearer.

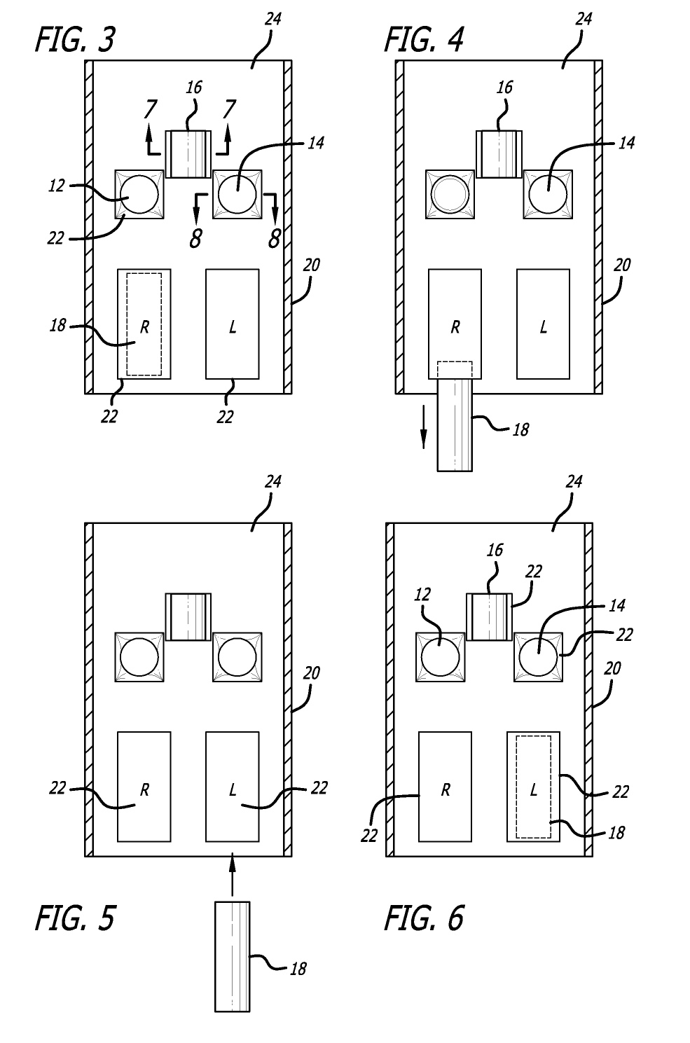

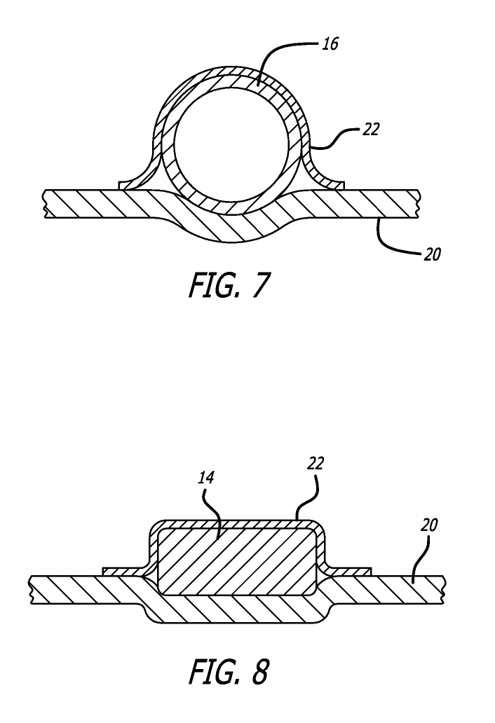

Referring now to FIGS. 3-8, the placement of the shock-absorbing elements 12 and 14 along with the pressure transmitting elements 16 and 18 on the inner surface 26 of the sleeve-like body 20 is shown. Each of these components is placed in a pre-formed pocket 22. The pressure transmitting element 18 can be placed in either of two pockets formed on the body 20. As can be seen in FIGS. 3-6, one pocket 22 is marked with the letter “R” and an adjacent pocket 22 is marked with the letter “L.” Since the tendon in the forearm is located at different locations on the right arm and left arm of the wearer, the placement of the pressure transmitting element 18 in the pocket marked with the “R” allows the wearer to wear the elbow brace 10 on the right arm. The location of the pocket marked “R” will properly place the pressure transmitting element 18 directly over the tendon of interest. If the wearer were to place this elbow brace on his/her left arm, with the pressure transmitting element 18 placed in the pocket marked “R”, then the element 18 would not be located over the tendon in the left arm. Rather, it would misaligned with the left tendon. The present invention allows the wearer to change the location of the pressure transmitting element 18 to the pocket marked “L” to allow the wearer to wear the elbow brace of his/her left arm. The shift of the pressure transmitting element 18 to the pocket marked “L” will now allow this element 18 to properly align over the tendon in the left arm. While pockets are used to house this element 18, it should be understood that other structures could be used to allow the wearer to change the location of this pressure transmitting element 18 as needed.

The shock-absorbing elements 12 and 14 are shown as solid elastic member which are sized to fit over the lateral and medial epicondyle regions. It should be appreciated that other shapes and sizes could be used in conjunction with these elements. For example, ring-like members made from a shock-absorbing material can be used. These shock-absorbing elements 12 and 14 are placed directly over the medial and lateral epicondyle such that the shock absorbent material actually surrounds the tip of the epicondyle and contacts the region around the tip where many of the tendons of the forearm are attached. The shock-absorbing characteristics of these first and second shock-absorbing elements helps transmit the shock away from the elbow region thus reducing the amount of trauma that can be caused to the tendons and tissue at the elbow region.

The pressure transmitting elements 16 and 18 are shown as generally hollow, elongate tubular members which are made from a plastic material, or similar materials which allow for some shock-absorbing capabilities as well. These shock-absorbing elements produce an inner radial force onto the tendons of interest and are again held in place by the sleeve-like body 20. These pressure transmitting elements each provide a certain amount of surface area which contacts the arm of the player and defines an area of contact which allows the brace to direct focused pressure to the specific tendons of interest. The placement of the pressure transmitting elements, as well as the shock-absorbing elements, is critical since a misalignment of these elements of the arm will not allow the elements to perform the necessary functions to the particular body parts.

During exercise or play, it is possible for the elbow brace to start to move or shift on the arm, which can cause the elements to move from their intended locations on the arm. The wearer merely has to open the first strap 36 and re-adjust the positioning strap 26 to again realign the shock-absorbing elements on the epicondyle (a simple and quick task) resulting in proper alignment of all components. The placement of the pressure transmitting elements is fixed with respect to the shock-absorbing elements via the sleeve-like member to thus provide the correct placement of these elements on the arm whenever the shock-absorbing elements are aligned. As a result, the player need not worry about specially aligning the pressure transmitting elements during play.

The shock-absorbing elements can be generally made from a rubber or rubber-like material. For example, the elements can have hardness substantially within the range of about 40 to 60 Shore hardness. If the material density or hardness is too little, then the shock energy again will not be sufficiently absorbed and dissipated. It should be appreciated that the materials need not be rubber but can be any material, such as elastomeric material, which provides the appropriate amount of shock-absorbing capability. Again, the critical factor is that the epicondyle be placed in contact with a shock-absorbing material which will dissipate a good portion of the shock energy that may be transmitted through the forearm to the elbow region.

From the above, it is evident that the present invention provides a novel approach for an elbow brace for preventing and attenuating the symptoms and pain associated with the condition known as tennis elbow. The elbow brace described above is superior over the prior art since the present invention provides both shock-absorbing and pressure transmitting capabilities to the body parts which are most affected by the shock energy and the stretching of the tendons which causes tennis elbow along with a quick and simple means for properly positioning these various components over the specific regions of the wearer's arm. While particular forms of the invention have been described and illustrated, it will also be apparent to those skilled in the art that various modifications may be made without departing from the spirit and scope of the invention. Accordingly, it is not intended that the invention be limited except by the appended claims.

I claim:

1. An elbow brace for preventing or attenuating tennis elbow comprising:
a first shock-absorbing element adapted to be placed in contact with the arm of the wearer directly over the medial epicondyle of the user;
a second shock-absorbing element adapted to be placed in contact with the arm of the wearer directly over the lateral epicondyle of the wearer;
a first pressure transmitting element adapted to be placed in contact with the arm of the wearer so as to apply focused pressure to the tendon that extends from the triceps brachii muscle and is attached to the olecranon of the wearer;
a second pressure transmitting element adapted to be placed in contact with the arm of the wearer so as to apply focused pressure to the tendon that extends from the extensor digitorum muscle and is attached to the humerus of the wearer;
a sleeve-like body housing the first and second shock-absorbing elements and the first and second pressure transmitting elements the sleeve-like body having at least one positioning strap associated with it to allow the user to open at least a portion of the sleeve-like body to allow the wearer to visualize the location of at least one of the shock-absorbing elements so as to help position the at least one shock-absorbing element on the arm of the wearer, the positioning strap having a fastening device for closing the sleeve-like body;
a first pressure generating strap associated with the sleeve-like body for applying inward radial pressure on the arm of the wearer, the first pressure generating strap being disposed over the positioning strap; and
a second pressure generating strap associated with the sleeve-like body for applying an inward radial force on the arm of the user.

2. The elbow brace as defined in claim 1, wherein the second pressure generating strap extends over the second pressure transmitting element and cooperates with the second pressure transmitting element to generate focused pressure to the tendon of the user.

3. The elbow brace as defined in claim 2, wherein the first pressure generating strap device extends over the first pressure transmitting element and cooperates with the first pressure transmitting element to generate focused pressure to the tendon of the user.

4. The elbow brace as defined in claim 3, wherein the sleeve-like body applies pressure to each of the first and second shock-absorbing elements when placed on the arm of the wearer.

5. The elbow brace as defined in claim 4, wherein each of the first and second strap devices are adapted to be positioned to achieve the desired inward radial force on the arm of the wearer.

6. The elbow brace as defined in claim 1, wherein the positioning strap is adapted to exert inward radial pressure on the arm of the wearer.

7. The elbow brace as defined in claim 1, wherein the sleeve-like body includes an inner surface and an outer surface with the first and second pressure transmitting elements and the first and second shock-absorbing elements being disposed on the inner surface.

8. The elbow brace as defined in claim 7, further including a first pocket disposed on the inner surface of the sleeve-like body and a second pocket spaced apart from the first pocket, the first pocket being used to house the second pressure transmitting element when the elbow brace is worn on the right arm of the wearer and the second pocket and the second pocket being used to house the second pressure transmitting element when the elbow brace is worn on the left arm of the wearer.

9. The elbow brace as defined in claim 1, wherein the first and second pressure generating straps include a quick release fastener attached thereto.

10. An elbow brace for preventing or reducing tennis elbow comprising:
a sleeve-like body having a plurality of pockets defined therein, the sleeve-like body having a closed position in which a portion of the sleeve-like body forms an encircling tubular shape and an open position in which the portion of the sleeve-like body forming the encircling tubular shape is discontinuous;
a first shock-absorbing element disposed within one of the pockets and in contact with the arm of the user directly over the user's medial epicondyle to absorb at least a portion of shock energy that may travel along the user's forearm in order to reduce the amount of shock imparted to the tendons attached to the medial epicondyle;
a second shock-absorbing element disposed within one of the pockets and in contact with the arm of the user directly over the user's lateral epicondyle to absorb at least a portion of shock energy that may travel along the user's forearm in order to reduce the amount of shock imparted to the tendons attached to the lateral epicondyle;
a first pressure transmitting element disposed within one of the pockets and adapted to be placed in contact with the arm of the wearer so as to apply focused pressure to the tendon that extends from the triceps brachii muscle and is attached to the olecranon of the wearer;
a second pressure transmitting element disposed within one of the pockets and adapted to be placed in contact with the arm of the wearer so as to apply focused pressure to the tendon that extends from the extensor digitorum muscle and is attached to the humerus of the wearer; and
a position strap associate with the sleeve-like body to move the sleeve-like body between the open and closed positions.

11. The elbow brace as defined in claim 10, further including a quick release fastener attached to the position strap.

12. The elbow brace as defined in claim 10, further including a first pressure generating strap associated with the sleeve-like body for applying inward radial pressure on the arm of the wearer, the first pressure generating strap being disposed over the positioning strap.

13. The elbow brace as defined in claim 12, further including a second pressure generating strap associated with the sleeve-like body for applying an inward radial force on the arm of the wearer.

14. The elbow brace as defined in claim 19, wherein the second pressure generating strap extends over the second pressure transmitting element and cooperates with the second pressure transmitting element to generate focused pressure to the tendon of the user.
